(19) United States
(12) Patent Application Publication
(10) Pub. No.: US 2022/0226409 A1
(43) Pub. Date: Jul. 21, 2022

(54) COMPOSITION FOR STIMULATING OF MYOGENESIS AND PREVENTION OF MUSCLE DAMAGE CONTAINING GINSENG EXTRACT

(71) Applicant: FAMENITY CO., LTD., Uiwang-si (KR)

(72) Inventors: Ji Won LEE, Gwacheon-si (KR); Jun Kee Hong, Yongin-si (KR)

(21) Appl. No.: 17/576,744

(22) Filed: Jan. 14, 2022

(30) Foreign Application Priority Data
Jan. 18, 2021 (KR) 10-2021-0006915

(51) Int. Cl. A61K 36/258; A61P 21/00; A23L 33/00; A23L 33/105

(52) U.S. Cl. CPC A61K 36/258; A61P 21/00; A23V 2002/00; A23L 33/105; A61K 2236/333; A23L 33/40

(57) ABSTRACT

The present disclosure relates to a composition for stimulating of myogenesis and prevention of muscle damage, comprising a ginseng extract. More specifically, the present disclosure provides a composition for stimulating of myogenesis and prevention of muscle damage, comprising non-saponin components of ginseng, which may rapidly recover the damaged muscles by stimulating myogenesis and increasing an expression of proteins related with a muscle control.

### CROSS-REFERENCE TO RELATED APPLICATIONS

This application claims the priority of the Korean Patent Applications NO 10-2021-0006915 filed on Jan. 18, 2021, in the Korean Intellectual Property Office, the disclosure of which is incorporated herein by reference.

### TECHNICAL FIELD

The present disclosure relates to a composition for stimulating of myogenesis and prevention of muscle damage, comprising a ginseng extract. More specifically, the present disclosure relates to a composition for stimulating of myogenesis and prevention of muscle damage, comprising non-saponin-based components of ginseng, which may rapidly recover the damaged muscles by stimulating myogenesis and increasing an expression of proteins related with a muscle control.

### BACKGROUND ART

Muscles are the largest tissues in the human body. Securing the proper muscle mass of the human body is essential for maintaining the structure of the human body, allowing each organ of the human body to perform its own function, and preventing various diseases.

Muscles are largely divided into smooth muscle, cardiac muscle, and skeletal muscle. Skeletal muscle is an organ that occupies a significant portion of the entire body and accounts for 40 to 50% of the total body weight, and promotes skeletal movement. These skeletal muscles do not divide, and are made up of multinucleated muscle fibers, which are created during embryogenesis. After the embryogenesis is completed, muscle is formed by the process of postnatal growth or myogenesis. In addition, myogenesis occurs even when muscles are damaged by frostbite, distortion, bruises, or the like.

In the myogenesis process, first, satellite cells are activated, and the activated satellite cells are differentiated into myoblasts. Differentiated myoblasts divide and fusion of myoblasts occurs to develop into myotubes, these myotube cells gather to form muscle fibers, and the muscle fibers form bundles to finally form a muscle.

The myogenesis process is regulated by various muscle regulatory factors such as MyoD, Myf5 (myogenic factor 5), myogenin, and MRF4 (myogenic regulator factor 4), and MyoD initiates the expression of muscle-specific genes such as myosin H chain (myosin heavy chain, MHC) and muscle creatine kinase (MCK) and induces satellite cells to differentiate into myoblasts, and induction of myogenin expression by the activity of MyoD is the most important factor in the fusion of myoblasts and is involved in the formation of myotube cells.

Meanwhile, myokine is an active material expressed or synthesized from skeletal muscle in response to muscle contraction, and functions in an autocrine, paracrine, or endocrine manner, and is also known to regulate functions of other tissues as well as muscles. Representative myokines include myostatin (MSTN), interleukin-6 (IL-6), irisin, etc.

Myostatin is a gene belonging to the transforming growth factor β (transforming growth factor-β, TGF-β) group, and is a protein that directly acts on muscle cells to inhibit myogenesis and differentiation of myocytes. In several previous studies, when the myostatin gene was knocked down or knocked out, muscle hypertrophy and insulin resistance were reduced, and as the expression level of myostatin decreased during exercise, the muscle size is known in be increased.

In addition, the degradation process of MyoD, a transcription factor essential to express myofibril protein important for myogenesis, is known to induce muscle atrophy by increasing protein degradation mediated by the UPP (ubiquitin proteasome pathway) process activated by the myostatin (MSTN) activin type II receptor (ActRIIB)-Smad2 pathway.

Meanwhile, if a problem occurs during the myogenesis process, such as differentiation from aged cells or satellite cells to myoblasts, division of myoblasts, etc. several muscle disorders and diseases such as muscle atrophy, myopathy, muscle injury, muscular dystrophy, sarcopenia, myoneural conductive disease, and nerve injury may occur.

In addition, sarcopenia, which is one of muscle diseases caused by problems occurring during the myogenesis process, may also appear due to cachexia. Cachexia is a type of chronic wasting complex syndrome and refers to a highly debilitating symptom that may be seen in the terminal stages of cancer, tuberculosis, hemophilia, etc. In particular, it occurs along with chronic diseases such as malignant tumors, chronic obstructive pulmonary disease, chronic heart failure, etc., and weight loss accompanied by anorexia, reduction of muscle mass and body fat, inflammatory response, etc. appear.

Muscle loss due to cachexia appears as a complex syndrome caused by a continuous decrease in skeletal muscle mass and functional impairment, and unlike the muscle loss disease caused by aging and myogenesis disorder, in which muscle mass loss occurs gradually and slowly, acute muscle loss symptoms appear. These differences in physiological characteristics also show differences in prevention and treatment.

Therefore, even if a sign of muscle loss occurs, treatment tailored to the characteristics of cachexia, aging, and myogenesis disorder is required depending on the cause of the occurrence.

Meanwhile, as a method for overcoming myogenesis disorders and diseases, a method of regenerating muscle cells has been recently reported, and it is known that this regeneration of muscle cells stimulates satellite cells existing outside the muscle cells to cause a division of the satellite cells to form muscle tissue. It has also been reported that the regeneration of muscle cells may be applied not only to the repair of damaged muscles, but also to natural muscle loss due to aging.

In addition, research results have been reported that the expression of MyoD and myogenin, which are important muscle regulators in the myogenesis process, is reduced in the course of cancer cachexia, and in a mouse model of muscle wasting (cachexia) caused by cancer or AIDS, research results have also been reported that the expression of myogenin and myosin, which are important muscle regulators in the myogenesis process, is reduced. In addition, as research results have been reported that the inhibition of myostatin affects muscle volume and function in a mouse model of cancer cachexia induced, it is expected that cachexia treatment will be possible by promoting myogenesis.

Therefore, as the importance of improving muscle health due to reduction in muscle loss and myogenesis is emerging, the search for functional materials is also being actively performed.

### RELATED ART DOCUMENT

### [Patent Document]

(Patent document 1) KR 10-2018-0059749 B1

(Patent document 2) KR 10-2019-0010337 B1

### DISCLOSURE

### Technical Problem

The object of the present disclosure is to provide a composition for stimulating of myogenesis and prevention of muscle damage, comprising a ginseng extract.

The object of the present disclosure is to provide a composition for stimulating of myogenesis and prevention of muscle damage, comprising non-saponin-based components of ginseng, which may rapidly recover the damaged muscles by stimulating of myogenesis and increasing an expression of proteins related with a muscle control.

### Technical Solution

In order to achieve the above object, the composition for stimulating of myogenesis and prevention of muscle damage according to an embodiment of the present disclosure comprises a ginseng extract, and the ginseng extract is a non-saponin-based compound separated and purified from ginseng.

The ginseng is selected from the group consisting of Korean ginseng (Panax ginseng), Whogi ginseng (P. quiquefolius), Chunchil ginseng (P. notoginseng), Jukgel japonicus (P. japonicus), Samyeop ginseng (P. trifolium), himalayan ginseng (P. pseudoginseng), Vietnamese ginseng (P. vietnamensis), American ginseng (P. quinquefolium) and a mixture thereof.

The ginseng extract is a concentrate concentrated under reduced pressure comprising a non-saponin-based compound, and the ginseng extract is separated into water layer by concentrating the ethanol extract of the ginseng under a reduced pressure, and removing the ethanol, butanol is added to the separated water layer and mixed, and then the mixture is separated into an upper layer and a lower layer, each of the upper layer and the lower layer is separated in the form of a concentrate concentrated under the reduced pressure, and the lower layer separated and concentrated under the reduced pressure is a water layer comprising a non-saponin-based compound.

The muscle damage is selected from the group consisting of muscle strain, muscle rupture, muscle tearing, contusion, distortion, rotator cuff syndrome, and myositis.

The food composition according to another embodiment of the present disclosure comprises the composition for stimulating of myogenesis and prevention of muscle damage.

The pharmaceutical composition according to another embodiment of the present disclosure comprises the composition for stimulating of myogenesis and prevention of muscle damage.

Hereinafter, the present invention will be described in more detail.

In the present disclosure, “muscle” comprehensively refers to tendons, muscles, and tendons, and “muscular function” means the ability to exert force by contraction of the muscle, and comprises the muscle strength which is an ability of exerting maximum contractile force to overcome resistance, muscular endurance, which is the ability representing whether how long or how many times a muscle can repeat contractions and relaxations with a given weight, and instantaneous strength, which is the ability to exert strong force in a short period of time. This muscle function is proportional to the muscle mass, and “improving muscle function” means making the muscle function improved better.

In the present disclosure, “antioxidation” means the efficacy exerted by inhibiting the generation of reactive oxygen species. The reactive oxygen species means an unstable state in which oxygen has free radicals, and thus has characteristics for having strong activity. Reactive oxygen species are generated by various physical, chemical and environmental factors such as enzyme system in the body, reduction metabolism, chemicals, pollutants, photochemical reactions, etc. In addition, it is known to cause various diseases comprising cellular aging or cancer by non-selective and irreversible destruction of lipids, proteins, sugars, DNA, etc., which are cellular components. It has been reported that when reactive oxygen species are excessive, it causes toxicity to the living body, that is, oxidative stress.

The composition for stimulating of myogenesis and prevention of muscle damage according to an embodiment of the present disclosure comprises a ginseng extract, and the ginseng extract is a non-saponin-based compound separated and purified from ginseng.

The ginseng (Panax ginseng C.A. Mayer) is a plant belonging to the genus Araliaceae botanically, and has been used medicinally from B.C. in China. In Korea, it has been used for trade and medicinal purposes since the Three Kingdoms period, and is still widely used as a herbal medicine or health food in various fields up to now.

Ginsenoside, the main functional ingredient of ginseng, contains about 3 to 6% of prosaponin, and among various saponins in the plant system, only ginseng saponin is specifically named, and currently, more than total 150 types of ginsenoside have been found. It has been found that ginsenoside has pharmacological efficacy such as central nervous system suppression and mental stability, pain relief, memory improvement, liver injury report, protein and lipid synthesis promotion, anti-diabetes, anti-stress, anti-oxidative active substance production promotion, immune regulation, platelet aggregation inhibition, anti-aging action etc., in addition to anti-cancer, anti-allergy, and anti-inflammatory, and the efficacy is different depending on the type of ginsenoside.

Ginsenoside of ginseng is connected to the high molecular component, so it is not easily absorbed into the body after ingestion, and thus is absorbed into the body after being ingested by a microorganism residing in the gut. That is, the ginsenoside form in which the sugar bound to the ginsenoside is decomposed is finally absorbed into the body, and each of the ginsenosides exhibits different physiological activities.

It is known that ginsenoside Rg1 and Rb1 enhance central nervous system activity, ginsenoside Re, Rg1 and panaxan A and B are good for diabetes, ginsenoside Re and Rg1 promote angiogenesis, and ginsenosides Rg3 and Rh2 exhibit anticancer efficacy. Recent studies have found that several ginseng extracts are effective against cachexia, fatigue, or muscle atrophy.

However, as it was found that polyacetylene, a non-saponin-based component of ginseng in addition to the saponin, inhibited cancer cell proliferation, studies on the physiological activity of effective ingredients other than saponin were conducted.

Non-saponin-based components of ginseng are physiologically active substances, and polyacetylenes, phenolic compounds, acidic polysaccharides, peptides, alkaloids, and amino acid derivatives have been found. In addition, other components of ginseng comprise volatile oil, sugar, starch, pectins, minerals, and the like.

However, there have been hardly any experiments on investigation of the stimulating of myogenesis and prevention of muscle damage for non-saponin-based components of ginseng, and the effect of ginseng as a drug.

Therefore, the ginseng extract of the present disclosure is a non-saponin-based compound separated and purified from ginseng, and has the effect for stimulating of myogenesis and prevention of muscle damage with the non-saponin-based component of ginseng.

The ginseng is selected from the group consisting of Korean ginseng (Panax ginseng), Whogi ginseng (P. quiquefolius), Chunchil ginseng (P. notoginseng), Jukgel japonicus (P. japonicus), Samyeop ginseng (P. trifolium), himalayan ginseng (P. pseudoginseng), Vietnamese ginseng (P. vietnamensis), American ginseng (P. quinquefolium) and a mixture thereof.

The ginseng may be applied to the present disclosure without limitation if it is the plant of the genus Ginseng. For example, the ginseng comprises any one selected from the group consisting of Korean ginseng (Panax ginseng), Whogi ginseng (P. quiquefolius), Chunchil ginseng (P. notoginseng), Jukgel japonicus (P. japonicus), Samyeop ginseng (P. trifolium), himalayan ginseng (P. pseudoginseng), Vietnamese ginseng (P. vietnamensis), American ginseng (P. quinquefolium) and a mixture thereof, but is not limited thereto. Preferably, the ginseng used in the present disclosure is Korean ginseng (Panax ginseng).

The composition stimulates differentiation of muscle cells by increasing the expression level of myogenin, a muscle regulator.

In the differentiation of muscle cells, satellite cells are activated to differentiate into myoblasts, fusion of myoblasts occurs to form myotubes, and then the myotubes form muscle fibers, and the muscle fibers bundled together to form muscles, wherein various muscle regulatory factors such as MyoD, Myf5 (myogenic factor 5), myogenin, and MRF4 (muscle regulatory factor 4), and muscle-specific factors such as myosin H chain (myosin heavy chain, MHC), muscle creatine kinase (MCK) are involved.

The myogenesis process is regulated by various muscle regulatory factors such as MyoD, Myf5 (myogenic factor 5), myogenin, and MRF4 (myogenic regulator factor 4), and MyoD initiates the expression of muscle-specific genes such as myosin H chain (myosin heavy chain, MHC) and muscle creatine kinase (MCK) and induces satellite cells to differentiate into myoblasts, and induction of myogenin expression by the activity of MyoD is the most important factor in the fusion of myoblasts and is involved in the formation of myotube cells.

Therefore, the composition for stimulating of myogenesis and prevention of muscle damage of the present disclosure may stimulate the differentiation of muscle cells by increasing the expression level of myogenin.

On the other hand, myokine is an active material expressed or synthesized from skeletal muscle in response to muscle contraction, and functions in an autocrine, paracrine, or endocrine manner, and is also known to regulate functions of other tissues as well as muscles. Representative myokines include myostatin (MSTN), interleukin-6 (IL-6), irisin, etc.

The myostatin is a gene belonging to the transforming growth factor β (TGF-β) group, and is a protein that acts directly on muscle cells to inhibit myogenesis and muscle cell differentiation.

The myostatin is known to be overexpressed in various diseases such as aging, muscular dystrophy, amyotrophic lateral sclerosis, chronic obstructive pulmonary disease, chronic heart failure, AIDS, cancer cachexia, renal failure, uremia, rheumatoid arthritis, etc.

The ginseng extract is a concentrate concentrated under reduced pressure comprising a non-saponin-based compound, and the ginseng extract is separated into water layer by concentrating the ethanol extract of the ginseng under a reduced pressure, and removing the ethanol, butanol is added to the separated water layer and mixed, and then the mixture is separated into an upper layer and a lower layer, each of the upper layer and the lower layer is separated in the form of a concentrate concentrated under the reduced pressure, and the lower layer separated and concentrated under the reduced pressure is a water layer comprising non-saponin-based compounds.

The ginseng ethanol extract is extracted using an extraction solvent selected from the group consisting of lower C₁-C₆ alcohols and a mixture thereof.

Specifically, the ginseng ethanol extract may be obtained by the following steps: washing the ginseng; drying the ginseng; pulverizing the ginseng; leaching the pulverized product by using an alcohol solvent; drying the leached sample; leaching the sample with water; and performing leaching.

The ginseng extract extracted by using the alcohol solvent may further comprise the step of performing fractionation using an alcohol solvent.

The method for preparing the extract may be a conventional extraction method in the art, such as an ultrasonic extraction method, a leaching method, a reflux extraction method, etc. Specifically, it may be an extract obtained by extracting a natural product from which foreign substances have been removed by washing and drying with an alcohol having 1 to 6 carbon atoms or a mixed solvent thereof, and may be an extract extracted by sequentially applying the solvents to the sample.

The reflux extraction method is carried out by 10 to 30 g of a pulverized product of a natural product and 50 to 100% of alcohol having 1 to 6 carbon atoms, based on 100 mL of alcohol having 1 to 6 carbon atoms for reflux time of 1 to 3 hours. More specifically, it is carried out by 10 to 20 g of a pulverized product of a natural product and 70 to 90% of an alcohol having 1 to 4 carbon atoms for a reflux time of 1 to 2 hours, based on 100 mL of an alcohol having 1 to 6 carbon atoms.

The leaching method is carried out at 15 to 30° C. for 24 to 72 hours, and 50 to 100% of alcohol having 1 to 6 carbon atoms is used as an extraction solvent. More specifically, it is carried out at 20 to 25° C. for 30 to 54 hours, and the extraction solvent is 70 to 80% of alcohol having 1 to 6 carbon atoms.

The ultrasonic extraction method is performed at 30 to 50° C. for 0.5 to 2.5 hours, and the extraction solvent is 50 to 100% of alcohol having 1 to 6 carbon atoms. Specifically, the extraction is performed at 40 to 50° C. for 1 to 2.5 hours, and 70 to 80% of an alcohol having 1 to 6 carbon atoms is used as an extraction solvent.

The extraction solvent may be used in an amount of 2 to 50 times, more specifically, 2 to 20 times, based on the weight of the sample. For extraction, the sample may be left for 1 to 72 hours and more specifically for 24 to 48 hours, for leaching in the extraction solvent.

After obtaining the extract or fraction, a method such as concentration or freeze-drying may be additionally used.

Muscle damage applied to the composition according to the present disclosure for stimulating of myogenesis and prevention of muscle damage is selected from the group consisting of muscle strain, muscle rupture, muscle tearing, contusion, distortion, rotator cuff syndrome, and myositis.

If a problem occurs during the myogenesis process, such as differentiation from aged cells or satellite cells to myoblasts, division of myoblasts, etc. several muscle disorders and diseases such as muscle atrophy, myopathy injury, muscular dystrophy, sarcopenia, myoneural conductive disease, and nerve injury may occur.

In addition, muscle damage and sarcopenia, which is one of muscle diseases caused by problems occurring during the myogenesis process, may also appear due to cachexia. Cachexia is a type of chronic wasting complex syndrome and refers to a highly debilitating symptom that may be seen in the terminal stages of cancer, tuberculosis, hemophilia, etc. In particular, it occurs along with chronic diseases such as malignant tumors, chronic obstructive pulmonary disease, chronic heart failure, etc., and weight loss accompanied by anorexia, reduction of muscle mass and body fat, inflammatory response, etc. appear.

Genital muscle diseases may be selected from the group consisting of muscular atrophy, myopathy, muscle injury, muscular dystrophy, myasthenia, sarcopenia, myoneural conductive disease, dermatomyositis, diabetic amyotrophy, nerve injury, amyotrophic lateral sclerosis (ALS), cachexia and degenerative muscle diseases and the cachexia may be AIDS (acquired immune deficiency syndrome, AIDS), celiac disease, chronic obstructive pulmonary disease (COPD), multiple sclerosis, rheumatoid arthritis, chronic heart failure, congenital heart failure, uremia, tuberculosis, Crohn's disease, untreated or severe type 1 diabetes, anorexia nervosa and cachexia caused by a lack of hormones.

Muscle damage and muscle loss due to cachexia appear as a complex syndrome caused by a continuous decrease in skeletal muscle mass and functional impairment, and unlike the muscle loss disease caused by aging and myogenesis disorder, in which muscle mass loss occurs gradually and slowly, acute muscle loss symptoms appear.

Therefore, the composition for stimulating of myogenesis and prevention of muscle damage of the present disclosure is to prevent loss of muscle mass and prevent muscle damage by the stimulating of myogenesis.

The composition suppresses muscle damage by reducing MuRF1 (Muscle ring finger 1) and foxo3a involved in protein degradation.

The MuRF1 (Muscle ring finger 1) is involved in the decomposition of myosin heavy chain (MyHC), which is a major component of the muscle, and the MuRF1 promoter comprises almost perfectly symmetrical (palindromic) glucocorticoid response element (GRE), Forkhead box O (FoxO) binding element (FBE) and a nuclear factor kappa B response element. This indicates that the FoxO factor is important for regulating the transcription of MuRF1. Therefore, inhibiting the activity of these genes may be an effective strategy for suppressing sarcopenia.

In addition, muscle mass is determined by the dynamic balance between anabolic and catabolism, and this has been reported that muscle atrophy through a variety of stimuli, including interleukin-1 (IL-1), tumor necrosis factor (TNF-α), and glucocorticoids occurs. It is known that MuRF1, a muscle-specific E3 ligase, plays an important role in such muscle atrophy.

In particular, MuRF1 causes muscle damage with a remarkable increase in various diseases such as nerve damage, diabetes, sepsis, hyperthyroidism, cachexia caused by cancer, etc., when muscles are not moved for a long time.

The foxo3a is one of the Forhead box O family genes (FOXO1, FOX03a, FOXO4, FOXO6) known as longevity genes, and protein modification is made by various signals or stimuli to regulate transcriptional activity.

The foxo3a gene may play a role in activating transcription of MnSOD and catalase genes due to electron activity, removing reactive oxygen species, and delaying aging of organs and tissues by inhibiting gene damage.

Therefore, the composition for stimulating of myogenesis and prevention of muscle damage of the present disclosure may suppress muscle damage by reducing the activity of MuRF1 and foxo3a, and may exhibit antioxidant or aging improvement effects.

The antioxidant means the efficacy exerted by inhibiting the generation of reactive oxygen species, reactive oxygen species (ROS) is a reduction metabolite of oxygen generated in mitochondria, and may be produced abnormally when the balance of oxidation-reduction metabolism in the body is disrupted due to exposure to excessive stress, radiation, chemicals, etc., or deterioration of the antioxidant system. When oxidative stress is induced by increased ROS, severe damage of intracellular DNA, proteins, and lipids is caused. Among the causes of various muscle dysfunction diseases, it has been recently announced in many studies that oxidative stress caused by an increase in the production of reactive oxygen species causes muscle dysfunction diseases.

Preferably, the composition for stimulating of myogenesis and prevention of muscle damage may further comprise Philadelphus schrenkii Rupr. var. schrenkii extracts, Ulmus laciniata (Trautv.) mayr extracts, and Callicarpa shiraswana Makino extracts.

Philadelphus schrenkii Rupr. var. schrenkii is a deciduous shrub growing in valleys throughout Korea. It grows in a place with good drainage of soil, high ambient humidity, and abundant humus. The tree is about 2-4 m tall, and the leaves are alternate phyllotaxis, 7-13 cm long and 4-7 cm wide, with a green surface an almost no hairs, and the back side is light green with fine hairs and is egg-shaped. Branches are divided into two, small branches are brown and hairy, and biennial branches are gray and peeled. The flowers are white and fragrant with several flowers on a long stalk at the top or where the leaves are attached. The fruits are 0.6-0.9 cm long and 0.4-0.5 cm in diameter in September month and is in an oval shape. It is mainly used for ornamental purposes, and the young leaves are used for food.

Ulmus laciniata (Trautv.) mayr is a deciduous broad-leaved tree of the dicotyledonous bicotyledonous family, Elmaceae, and grows in the valley below the hillside. The height is 20 m and the diameter is about 1 m, and the small branches are light brown. The leaves are alternate, long oval or inverted egg-shaped, broad, with the edges deeply dented in 3 places and sharply pointed. Also, the tree is 10˜20 cm long, has sharp double serrated edges, has rough and hairy surface, and has a light green back. Flowers are bisexual and bloom from April to May, and inflorescences are divided into 5-6 pieces. 5-6 stamens are magenta, and the style is divided into two. The fruit is a citrus fruit, flat, 1.5 cm long, broad egg-shaped, and ripens in May to June. It is used as tools for furniture, and firewood, and the bark is used as materials for medicinal purposes.

Callicarpa shiraswana Makino grows in the forest at the foot of the island in the southern coast of Korea, and has weak cold resistance but strong sound resistance, so it grows well on the beach. Leaves are opposite, egg-shaped, oval or oval-lanceolate, pointed, round or acute, 3-12 cm long and 2.5-5 cm wide, with fine dotted spots on both sides, short hairs on the surface, dense hairs on the back side, and there is the edge having sharp teeth on the leaf, and the petiole is 5-10 mm long, and the virgin hairs are dense. Flowers are bloomed in August, and the inflorescence is axillary and densely clustered with virgin hairs, and calyxes are deeply divided into 5 pieces, and the hairs of virgin or pinnate are dense. The corolla is 4-5 mm long, light purple, and the plate tube is almost the same length as the calyx, and the stamen has the same length as the corolla.

More preferably, the composition for stimulating of myogenesis and prevention of muscle damage of the present disclosure comprises 10 to 20 parts by weight of the Philadelphus schrenkii Rupr. var. schrenkii extracts, 10 to 20 parts by weight of Ulmus laciniata (Trautv.) mayr extracts, and 10 to 20 parts by weight of Callicarpa shiraswana Makino extracts, based on 100 parts by weight of the ginseng extract.

According to the above range, the composition for stimulating of myogenesis and prevention of muscle damage is possible to elevate the reduction activity of MuRF1 and foxo3a, which are involved in the expression and proteolysis of myogenin, muscle regulatory factors, and may be provided as an excellent composition for stimulating of myogenesis and prevention of muscle damage.

In addition, as it is composed of a complex extract, the effect of the stimulating of myogenesis is equal or increased, compared to the case of using only the ginseng extract alone, and further the composition for stimulating of myogenesis and prevention of muscle damage may be provided since it may prevent the muscle damage.

The extract is extracted by using an extraction solvent selected from the group consisting of water, lower C₁-C₆ alcohols, and a mixture thereof.

Specifically, in order to prepare the extract, the natural extract may be obtained by comprising a step of washing the natural product; a step of drying after washing; a step of pulverizing the natural product after drying; a step of leaching the pulverized product using an organic solvent; a step of drying the sample after leaching; a step of leaching with water; and a step of leaching.

The natural extract extracted by using the organic solvent may further comprise a step of performing fractionation using an organic solvent.

The method for preparing the extract may be a conventional extraction method in the art, such as an ultrasonic extraction method, a leaching method, a reflux extraction method, etc. Specifically, it may be an extract obtained by extracting a natural product from which foreign substances have been removed by washing and drying with water, an alcohol having 1 to 6 carbon atoms or a mixed solvent thereof, and may be an extract extracted by sequentially applying the solvents to the sample.

The reflux extraction method is carried out by 10 to 30 g of a pulverized product of a natural product and 50 to 100% of alcohol having 1 to 6 carbon atoms, based on 100 mL of water or alcohol having 1 to 6 carbon atoms for reflux time of 1 to 3 hours. More specifically, it is carried out by 10 to 20 g of a pulverized product of a natural product and 70 to 90% of an alcohol having 1 to 4 carbon atoms or water for a reflux time of 1 to 2 hours, based on 100 mL of water or an alcohol having 1 to 6 carbon atoms.

The leaching method is carried out at 15 to 30° C. for 24 to 72 hours, and water or 50 to 100% of alcohol having 1 to 6 carbon atoms is used as an extraction solvent. More specifically, it is carried out at 20 to 25° C. for 30 to 54 hours, and the extraction solvent is water or 70 to 80% of alcohol having 1 to 6 carbon atoms.

The ultrasonic extraction method is performed at 30 to 50° C. for 0.5 to 2.5 hours, and the extraction solvent is water or 50 to 100% of alcohol having 1 to 6 carbon atoms. Specifically, the extraction is performed at 40 to 50° C. for 1 to 2.5 hours, and water or 70 to 80% of an alcohol having 1 to 6 carbon atoms is used as an extraction solvent.

The extraction solvent may be used in an amount of 2 to 50 times, more specifically, 2 to 20 times, based on the weight of the sample. For extraction, the sample may be left for 1 to 72 hours and more specifically for 24 to 48 hours, for extracting in the extraction solvent.

After extraction, the extract may be fractionated by sequentially applying a fresh fractionation solvent. The fractionation solvent used for fractionation is any one or more selected from the group consisting of water, hexane, butanol, ethyl acetic acid, ethyl acetate, methylene chloride and a mixture thereof, preferably ethyl acetate or methylene chloride.

After obtaining the extract or fraction, a method such as concentration or freeze-drying may be additionally used.

The food composition according to another embodiment of the present disclosure comprises the composition for stimulating of myogenesis and prevention of muscle damage.

As a specific example, it is possible to prepare a processed food capable of enhancing the effect for stimulating of myogenesis and prevention of muscle damage by using the food composition. Such processed foods comprise, for example, confectionery, beverages, alcoholic beverages, fermented foods, canned foods, milk processed products, processed meat foods, noodles, and the like. Confectionery comprises biscuits, pies, cakes, breads, candies, jellies, gums, cereals, and the like. The beverage comprises drinking water, carbonated beverage, functional ionized beverage, functional ionized beverage, juice (e.g., apple, pear, grape, aloe, tangerine, peach, carrot, tomato juice, etc.), sikhye, and the like. Alcoholic beverages comprise sake, whiskey, shochu, beer, Western liquor, fruit wine, and the like. Fermented foods comprise soy sauce, soybean paste, red pepper paste, and the like. Canned food comprises canned seafood (e.g., canned tuna, mackerel, saury, conch, etc.), canned livestock (canned beef, pork, chicken, turkey, etc.), and canned agricultural products (canned corn, peach, pineapple, etc.). Milk processed products comprises cheese, butter, yogurt, and the like. Processed meat products comprise pork cutlet, beef cutlet, chicken cutlet, sausage, sweet and sour pork, nuggets, Bulgogi and the like. Noodles such as sealed packaged raw noodles are comprised. In addition to these, the composition may be used in retort food, soup, and the like.

The pharmaceutical composition according to another embodiment of the present disclosure comprises the composition for stimulating of myogenesis and prevention of muscle damage.

The pharmaceutical composition may further comprise one selected from the group consisting of pharmaceutically acceptable carriers, excipients and diluents. Specifically, the pharmaceutical composition of the present disclosure may be formulated and used in the form of oral dosage such as powders, granules, tablets, capsules, suspensions, emulsions, syrups, aerosols, etc., external preparations, suppositories, and sterile injectable solutions, respectively, according to conventional methods. Carriers, excipients and diluents that may be comprised in the pharmaceutical composition comprise lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil. In the case of formulation, it is formulated using diluents or excipients such as fillers, extenders, binders, wetting agents, disintegrants, surfactants, etc., that are usually used. Solid formulation for oral administration comprises tablets, pills, powders, granules, capsules, and the like, and these solid formulations are prepared by mixing at least one excipient, for example, starch, calcium carbonate, sucrose or lactose, gelatin, etc. with the extracts and fraction thereof. In addition to simple excipients, lubricants such as magnesium stearate and talc are also used. A liquid formulation for oral administration may be suspensions, oral liquids, emulsions, syrups, and the like, and comprise various excipients, for example, a wetting agent, a sweetener, an aromatic, a preservative, and the like, in addition to water and liquid paraffin which are commonly used as a simple diluent. Formulation for parenteral administration includes sterilized aqueous solutions, non-aqueous solutions, suspensions, emulsions, lyophilized formulations, and suppositories. As the non-aqueous solvent and suspending agent, propylene glycol, polyethylene glycol, vegetable oil such as olive oil, and injectable ester such as ethyl oleate may be used. As a base for suppositories, witepsol, macrogol, tween 61, cacao butter, laurin, glycerogelatin, and the like may be used.

The pharmaceutical composition of the present disclosure may be administered in a pharmaceutically effective amount, and the term “pharmaceutically effective amount” of the present disclosure means an amount sufficient to treat or prevent a disease at a reasonable benefit/risk ratio applicable to medical treatment or prevention, and the effective dose level may be determined depending on the severity of the disease, the activity of the drug, the patient's age, weight, health, sex, the patient's sensitivity to the drug, the time of administration of the composition of the present disclosure used, the route of administration and the rate of excretion, duration for treatment, factors including drugs used in combination with or concomitantly with the composition of the present disclosure and other factors well known in the medical field. The pharmaceutical composition of the present disclosure may be administered alone or in combination with a known immunotherapeutic agent. In consideration of all of the above factors, it is important to administer an amount capable of obtaining the maximum effect with a minimum amount without side effects.

In addition, the composition for stimulating of myogenesis and prevention of muscle damage according to the present disclosure may exert antioxidant efficacy by inhibiting the generation of reactive oxygen species (ROS), and thus may be used as an antioxidant composition. Here, the antioxidant composition may be used for cosmetics, foodstuffs, pharmaceuticals or quasi-drugs, but is not limited thereto.

### Advantageous Effects

The present disclosure may provide a composition for stimulating of myogenesis and prevention of muscle damage, comprising a ginseng extract.

The present disclosure may provide a composition for stimulating of myogenesis and prevention of muscle damage, comprising non-saponin-based components of ginseng, which may rapidly recover the damaged muscles by the stimulating of myogenesis and increase of an expression of proteins related with a muscle control.

### BRIEF DESCRIPTION OF DRAWINGS

FIG. 1A, FIG. 1B, FIG. 1C and FIG. 1D are an experimental result showing the cytotoxicity of a ginseng extract according to an embodiment of the present disclosure.

FIG. 2A, FIG. 2B, FIG. 2C and FIG. 2D are an experimental result showing the cytoprotective effect of a ginseng extract according to an embodiment of the present disclosure.

FIG. 3 is a result confirming that myoblasts are morphologically differentiated into myotube cells through differentiation induction according to an embodiment of the present disclosure.

FIG. 4A and FIG. 4B are an experimental result showing an expression level of myogenin of a ginseng extract according to an embodiment of the present disclosure.

FIG. 5 is an experimental result showing the effect of ginseng extract according to an embodiment of the present disclosure on ROS generation against oxidative stress.

FIG. 6A, FIG. 6B, FIG. 6C, FIG. 6D and FIG. 6E are an experimental result for confirming an ability of inhibiting the muscle damage of the ginseng extract according to an embodiment of the present disclosure.

### BEST MODE

Hereinafter, embodiments of the present disclosures will be described in detail so that those skilled in the art can easily implement the present invention. However, the present disclosures may be implemented in various different forms and is not limited to the embodiments described herein.

### Preparation Example 1: Preparation of Ginseng Extract

1. Preparation of Ginseng Extract

For samples of Korean ginseng (domestic, K-23), Korean ginseng (China, G-3), Whogi ginseng (H-3) and Chunchil ginseng (J-3), the roots were ground into powder with a mixer, and was blocked from the light, sealed and stored at room temperature until use. In order to prepare a sample for efficacy evaluation, 50% of ethanol was added to the ginseng powder with shaking, and then heated to 60° C. and extracted for 4 hours. Thereafter, ethanol was removed using a rotary evaporator, and the same amount of butanol (Daejung, Korea) was added to the remaining water layer, mixed, and then the layers were separated.

After that, the water-saturated butanol layer (B) comprising saponins on the upper layer and water layer (W) comprising non-saponin-based components on the lower layer were separated and concentrated under reduced pressure, respectively, and each concentrate was collected, and the water-saturated butane layer referred to as K-23B, and the water layer referred to as K-23W, in Korean ginseng (domestic, K-23), the water-saturated butane layer referred to as G-3B, and the water layer referred to as G-3W in Korean ginseng (China, G-3), the water-saturated butane layer referred to as H-3B, and the water layer referred to as H-3W in Whogi ginseng (H-3), the water-saturated butane layer referred to as J-3B, and the water layer referred to as J-3W in Chunchil ginseng (J-3) were used as samples for efficacy evaluation.

### Experimental Example 1: Confirmation of Cytotoxic and Cytoprotective Effects of Ginseng Extract

In order to confirm effect of ginseng extract (K-23B, K-23W, G-3B, G-3W, H-3B, H-3W, J-3B and J-3W) samples on C2C12 cells according to ginseng's native location and extraction method, the evaluation of cytotoxicity and cell viability was performed as follows.

The evaluation of cytotoxicity and cell viability of the sample for C2C12 was measured using the Cell Counting Kit-8 (CK04-20) according to the manufacturer's test method.

1. Cell Culture

The mouse-derived C2C12 cell line was purchased from the American Type Culture Collection (ATCC, CRL-1772; Manassas, Va., USA) and cultured in a 5% CO₂ incubator at 37° C.

2. Cytotoxicity to Ginseng Extract

In order to confirm the cytotoxicity of the ginseng extract samples, C2C12 cells were seeded in a 96-well plate at a concentration of 1×10⁴ cells/ml the day before the experiment, and ginseng extracts according to a type (K-23B, K-23W, G-3B, G-3W, H-3B, H-3W, J-3B and J-3W) were treated with 12.5, 25, 50 and 100 μg/ml and incubated for 24 hours. After that, the CCK-8 solution was aliquoted by 10 μL, which corresponds to 10% of the total volume, and incubated for 2 hours, and then absorbance at 460 nm using a micro-plate reader (Infinite M200 PRO NanoQuant, TECAN, Zurich, Switzerland) was measured.

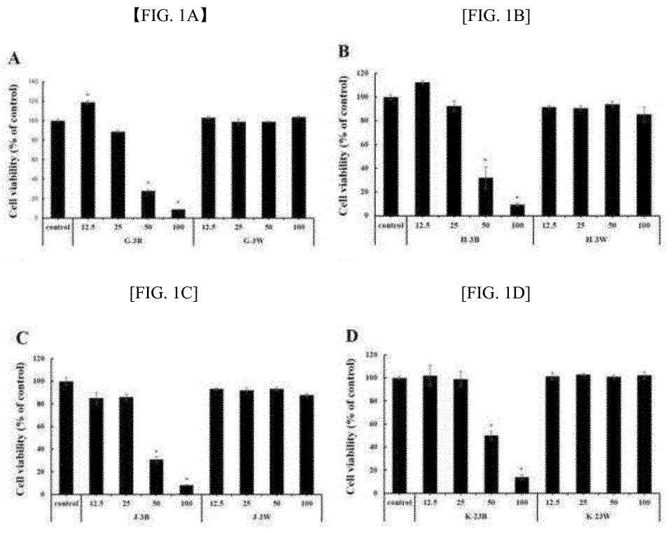

The results are shown in FIG. 1 below, and it was confirmed that in all of K-23B, G-3B, H-3B and J-3B, the saponin-containing water-saturated butane layer (B) group of the upper layer, cytotoxicity appeared according to an increase of the concentration.

On the other hand, it was confirmed that there was no significant change in cell viability in K-23W, G-3W, H-3W and J-3W of the water layer (W) group containing the non-saponin-based component.

Therefore, it was confirmed that K-23W, G-3W, H-3W and J-3W, the water layer (W) group containing the non-saponin-based component of the ginseng extract, do not have their own cytotoxicity, and the cell health is at a level similar to that of the control group.

3. Confirmation of Cytoprotective Effect on Ginseng Extract

In order to confirm the cytoprotective effect on the ginseng extract sample, C2C12 cells were divided to a control group, H₂O₂ (600 μM treatment group, and ginseng extract samples (K-23B, K-23W, G-3B, G-3W, H-3B, H-3W, J-3B and J-3W) 3.125, 6.25, 12.5 μg/mL+H₂O₂ (600 μM)) treatment groups.

As a result of treating each concentration of H₂O₂, cell viability was shown around 50% at 600 μM, so this study was conducted based on the above, and after pre-treatment with ginseng extract for 1 hour, it was treated with H₂O₂, a toxic substance, and cultured for 24 hours to confirm cell viability.

In order to confirm the protective efficacy, C2C12 cells were seeded in a 96-well plate at a concentration of 1×10⁴ cells/ml the day before the experiment, and the cells in ginseng extract treatment group were treated with a sample suitable for each concentration, and 1 hour later, except for the control group, all groups were treated with 600 μM H₂O₂. Then, after incubation for 24 hours, each 10 μL of CCK-8 solution was dispensed and cultured for 2 hours, and absorbance was measured at 460 nm using a micro-plate reader (Infinite M200 PRO NanoQuant, TECAN, Zurich, Switzerland).

H₂O₂ treatment in the C2C12 cells causes oxidative cell damage, and oxidative cell damage caused by H₂O₂ treatment in C2C12 cells was confirmed and the protective effect of ginseng extracts (K-23B, K-23W, G-3B, G-3W, H-3B, H-3W, J-3B and J-3W) was confirmed.

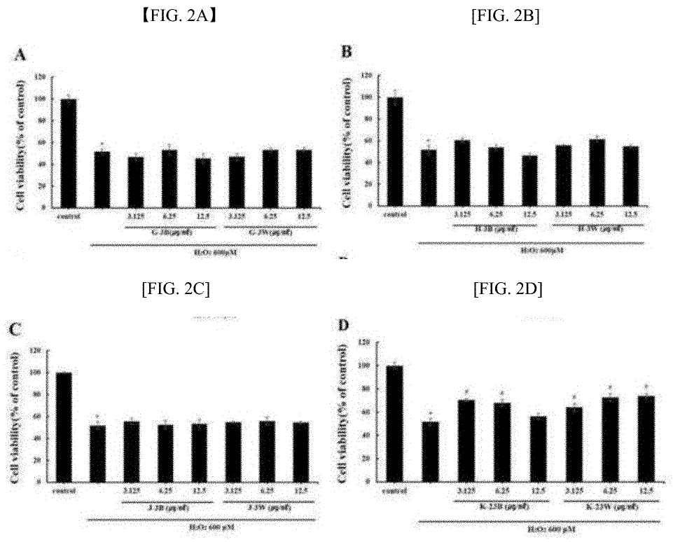

The results are shown in FIG. 2 below, and the G-3B, G-3W, H-3B, H-3W, J-3B and J-3W groups showed no significant difference in cell viability compared to the treatment only with the upper layer of H₂O₂, and in the case of the K-23B and K-23W groups, the cell viability was significantly higher, confirming a cytoprotective effect against oxidative stress caused by H₂O₂.

In particular, in the case of the water layer K-23W group containing non-saponin-based components, it was confirmed that the cells were protected from oxidative damage than the water saturated butane layer K-23B group containing saponin-based components depending on the concentration.

### Experimental Example 2: Capability of Myogenesis of Ginseng Extract

In order to determine whether the ginseng extract affects the differentiation of myoblasts into myotubes, C2C12 myoblast cells were induced to be differentiated into myotubes.

1. Differentiation Induction

Dulbecco's modified Eagle's medium (DMEM) containing 10% Fetal Bovine Serum (FBS) was used in the cell proliferation phase before differentiation of the cells cultured in experimental example 1, and during differentiation induction, 10% FBS was replaced with 2% horse serum (HS). An appropriate number of cells were maintained by subculturing every 48 hours in order to solve the overdensity phenomenon caused by the proliferation of cell numbers. During differentiation, C2C12 myoblast cells were seeded in a 6-well plate at a concentration of 1×10⁵ cells/ml, cultured until 70-80% confluence, and the differentiation medium was changed once a day to confirm differentiation in the form of myotubes under a microscope.

2. Protein Isolation and Western Blot Analysis

Western blot analysis was performed to confirm the differentiation of myoblasts into myotubes and the changes in factors affecting the cytoprotective effect for each sample.

After appropriate cell culture and treatment in 100 mm dish, the prepared intracellular protein was isolated by using 1 mM PMSF (phenylmethylsulfonylfluoride), 1% protease inhibitor cocktail, and NP40 cell lysis buffer (Invitrogen, Grand Island, N.Y.).

The isolated protein was quantified using the Pierce™ BCA Protein Assay Kit (Invitrogen). After electrophoresis of 30 μg of protein using Bolt™ 4-12% Bis-Tris Plus Gels, the protein was transferred to the dry iBlot® Transfer Stack (Invitrogen), a nitrocellulose membrane of the gel matrix by using the iBlot® Gel Transfer Device (Invitrogen). Each membrane was blocked at room temperature for 1 hour by using 5% skim milk and washed 3 times with 0.1% TBST buffer (TBS in 0.1% tween20). After the treatment with primary anti-myogenin antibody [F5D] (ab1835) (1:250, abcam), anti-MyoD1 antibody [5.2F] (ab16148) (1:1000, abcam), anti-MURF1 antibody (ab96857) (1:1000, abcam), anti-FOXO3A (phospho S253) antibody (ab47285) (1:1000, abcam), anti-FOXO3A antibody (ab12162) (1:2500, abcam), the reaction was conducted overnight at refrigerator at 4° C. The secondary antibody was reacted for 1 hour at room temperature using HRP-conjugated IgG (1:30000 dilution). After washing 3 times, the expression amount of protein was confirmed as a protein band developed by using a C-DiGit® Blot Scanner (LI-COR, Lincoln, Nebr., USA). The protein band was quantified by using the Image J Program (National Institutes of Health, USA), as represented with a fold value for the result value of the control group.

The results are shown in FIG. 3 below, and it could be confirmed that myoblasts were morphologically differentiated into myotubes through this differentiation induction, and in the case of treating differentiated cells with H₂O₂, the tube length was shortened and cells died.

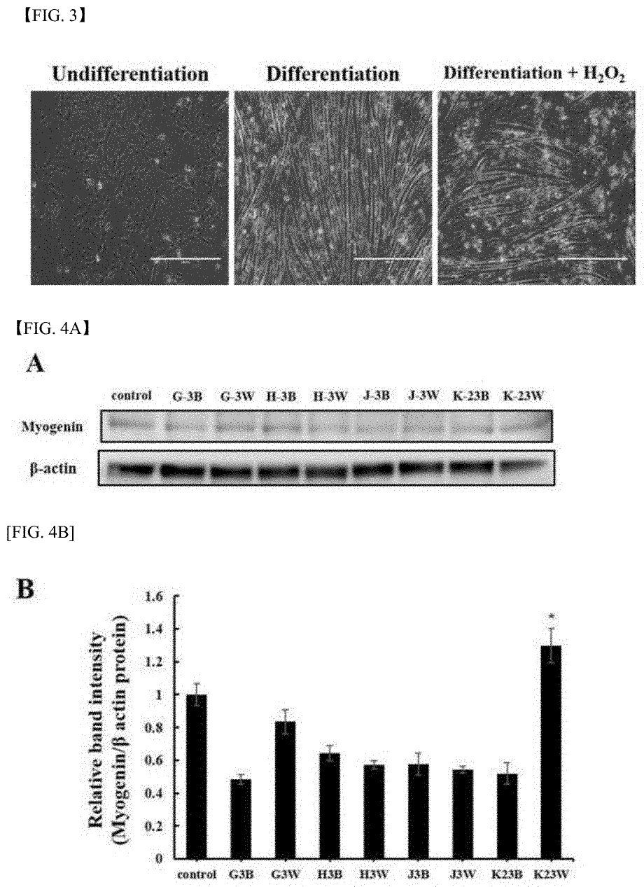

In addition, as shown in FIG. 4 below, differentiation was induced by using each sample (K-23B, K-23W, G-3B, G-3W, H-3B, H-3W, J-3B and J-3W), and as a result of confirming the expression of myogenin, a skeletal muscle differentiation induction marker gene, by western blot analysis, the W group showed a similar level of myogenin expression as the control compared with the B group.

In particular, in the case of K-23W, it was confirmed that the expression level of myogenin was increased, so that K-23W further stimulated the differentiation of muscle cells.

### Experimental Example 3: Effect of Ginseng Extract on ROS Generation

1. Measurement of ROS Generation

Intracellular ROS levels were measured by using the DCF-DA method. C2C12 myoblasts were dispensed into a 6-well plate at 1×10⁵ cells/mL, cultured for 24 hours, and then ginseng extract samples (K-23B, K-23W, G-3B, G-3W, H-3B, H-3W, J-3B and J-3W) were treated at a concentration of 12.5 μg/mL for 24 hours in serum free DMEM. After 1 hour, it was washed with PBS and treated with 600 μM of H₂O₂ for 24 hours. After washing with PBS, 10 μM DCF-DA was dispensed into each well and incubated in an incubator at 37° C. and 5% CO₂ conditions for 30 minutes. After 30 minutes, it was washed with PBS, and was dispensed into 1 mL of PBS into each well, and then the fluorescence at excitation 485/20 and emission 528/20 by using a micro-plate reader (Infinite M200 PRO NanoQuant, TECAN, Zurich, Switzerland) was measured.

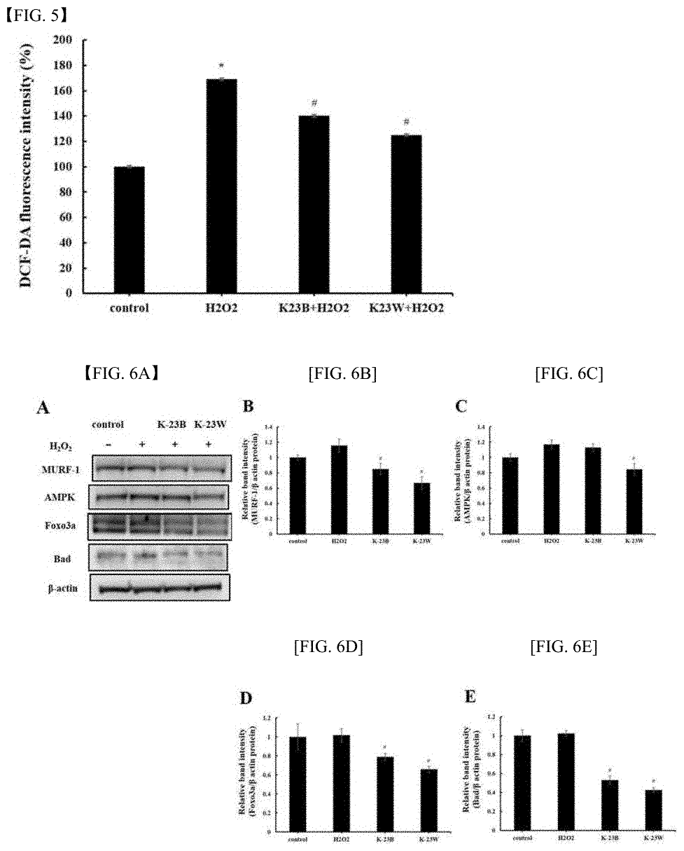

The results are shown in FIG. 5, and cell damage caused by H₂O₂ is related to damage of mitochondrial function caused by abnormal ROS in mitochondria, and the protective effect of the ginseng extract was confirmed.

Intracellular ROS levels were measured by using the DCF-DA method. As a result of measuring fluorescence, there was no significant difference in a change of fluorescence in the other extracts compared with the H₂O₂ group (Data not shown), but in the case of K-23, H₂O₂ was significantly higher than that of the control group, and as a result of treating the extract, it could be confirmed that the fluorescence was significantly reduced in K-23W compared to the group treated with H₂O₂. Through this, it was confirmed that the Korean ginseng (domestic, K-23) group among ginseng extracts had a protective effect against oxidative stress, and the K-23W group effectively reduced ROS compared to other extracts.

### Experimental Example 4: Capability of Inhibiting Muscle Damage of Ginseng Extract

AMPK and proteolytic signal transmitters MuRF1 and foxo3a, which are activated under stress due to AMP increase following depletion of ATP, were confirmed for muscle damage by using western blot. In addition, the protective effect of the extract against cell damage due to oxidative stress by inhibiting apoptosis was confirmed. Protein expression levels were confirmed mainly in Korean ginseng (domestic, K-23) and Korean ginseng (China, G-3) groups, which showed protective effects through cell viability.

In comparison with the control group, the protein expression levels of AMPK and MuRF1, which are proteolysis markers, were checked in the group treated with H₂O₂ to determine whether muscle damage occurred. As a result of H₂O₂ treatment, it was confirmed that the expression level of each protein was increased, compared with the control, and muscle damage occurred. In addition, as a result of treating each extract with H₂O₂, it could be confirmed that the protein degradation markers were decreased, compared to H₂O₂, but the difference was insignificant except for K-23W. In K-23W, it was confirmed that muscle damage is suppressed by somewhat inhibiting the activity of AMPK and decreasing MURF-a and Foxo3a. In addition, it was confirmed that by reducing Bad, an apoptosis-inducing factor, apoptosis was suppressed, thereby inhibiting a death of cell (FIG. 6).

### Preparation Example 2: Preparation of Complex Extract

1. Preparation of Philadelphus schrenkii Rupr. Var. Schrenkii Extracts

First, the roots of the Philadelphus schrenkii Rupr. var. schrenkii were thoroughly washed under running water and then completely dried naturally. The dried Philadelphus schrenkii Rupr. var. schrenkii was pulverized with a blender and then leached for 48 hours at room temperature using 70% ethanol, respectively, and then the sample was filtered to prepare the Philadelphus schrenkii Rupr. var. schrenkii extracts (PEs).

2. Preparation of the Other Extract

In the same manner as that of the Philadelphus schrenkii Rupr. var. schrenkii extracts (PEs), Ulmus laciniata (Trautv.) mayr root and Callicarpa shiraswana Makino root were used to prepare Ulmus laciniata (Trautv.) mayr extracts (UEs) and Callicarpa shiraswana Makino extracts (CEs).

3. Preparation of Complex Extract

Through examples 1 to 4 of the ginseng extracts (K-23B, K-23W, G-3B, G-3W, H-3B, H-3W, J-3B and J-3W) prepared in preparation example 1, water layer K-23W containing the non-saponin-based component, which was confirmed to have an excellent effect, was prepared as a complex extract by mixing it with Philadelphus schrenkii Rupr. var. schrenkii extracst (PEs), Ulmus laciniata (Trautv.) mayr extracts (UEs), and Callicarpa shiraswana Makino extracts (CEs) prepared in preparation example 2 in the content range as shown in Table 1 below.

TABLE 1

| | ME1 | ME2 | ME3 | ME4 | ME5 | ME6 |
|---|---|---|---|---|---|---|
| K-23W | 100 | 100 | 100 | 100 | 100 | 100 |
| PE | — | 5 | 10 | 15 | 20 | 25 |
| UE | — | 5 | 10 | 15 | 20 | 25 |
| CE | — | 5 | 10 | 15 | 20 | 25 |

(Unit: part by weight)

### Experimental Example 5: Effect of Complex Extract

In order to confirm the effect on the composition for stimulating myogenesis and preventing muscle damage of the present disclosure, an experiment was conducted in the same manner as in examples 1 to 4, and for comparison, K-23W, which showed an excellent effect, was given as an index of 5, and the effect was shown in Table 2 below.

TABLE 2

| | K-23W | | | | | |
|---|---|---|---|---|---|---|
| | (ME1) | ME2 | ME3 | ME4 | ME5 | ME6 |
| Cytoprotective effect | 5 | 5 | 5 | 5 | 5 | 5 |
| Capability of | 5 | 5 | 5 | 5 | 5 | 5 |
| myogenesis | | | | | | |
| Protective effect on | 5 | 5 | 6 | 6 | 5 | 5 |
| oxidative stress | | | | | | |
| Capability of inhibiting | 5 | 5 | 5 | 5 | 5 | 5 |
| muscle damage | | | | | | |

(Unit: Index)

According to Table 2, it was confirmed that ME2 to MEG mixing with Philadelphus schrenkii Rupr. var. schrenkii extracts (PEs), Ulmus laciniata (Trautv.) mayr extracts (UEs) and Callicarpa shiraswana Makino extracts (CEs), had an equal or higher effect, compared to ME1 comprising only K-23W, a water layer (W) group comprising the non-saponin-based components of Korean ginseng (domestic, K-23),

### Experimental Example 6: Palatability Evaluation

Palatability evaluation for the ME1 to MEG was conducted. After preparing ME1 to MEG as tea, and providing it to 20 adult men and women, evaluation of taste and aroma was requested.

The evaluation score was requested to be evaluated on a scale of 1 to 10 for each item, and the evaluation result was provided by converting it into an average score.

In the index, the higher the number, the higher palatability.

TABLE 3

| | K-23W | | | | | |
|---|---|---|---|---|---|---|
| | (ME1) | ME2 | ME3 | ME4 | ME5 | ME6 |
| Taste | 4.5 | 5.5 | 7.5 | 7.5 | 8.0 | 6.5 |
| Perfume | 4 | 4.5 | 7.0 | 7.5 | 8.0 | 5 |
| Total palatability | 4 | 5 | 7.0 | 7.5 | 8.0 | 5 |
| (average) | | | | | | |

(Unit: Index)

Referring to Table 3, it was confirmed that in K-23W (ME1), palatability was lowered due to the characteristic bitter taste and flavor of ginseng. However, it was confirmed that the palatability was excellent when other natural extracts of ME2 and ME6 were mixed and used.

In particular, in the case of ME3 and ME5, it was confirmed that they exhibit relatively excellent taste and perfume, and could be provided as a food composition with high palatability.

Although the preferred embodiments of the present disclosures have been described in detail above, the scope of the present invention is not limited thereto, and various modifications and improvements by those skilled in the art using the basic concept of the present invention defined in the following claims also belong to the scope of rights of the present invention.

1. A composition for stimulating of myogenesis and prevention of muscle damage, comprising a ginseng extract, wherein the ginseng extract comprises a non-saponin-based compound separated and purified from ginseng.

2. The composition for stimulating of myogenesis and prevention of muscle damage of claim 1,
wherein the ginseng is selected from the group consisting of Korean ginseng (Panax ginseng), Whogi ginseng (P. quiquefolius), Chunchil ginseng (P. notoginseng), Jukgel ginseng (P. japonicus), Samyeop ginseng (P. trifolium), Himalayan ginseng (P. pseudoginseng), Vietnamese ginseng (P. vietnamensis), American ginseng (P. quinquefolium) and mixture thereof.

3. The composition for stimulation of myogenesis and prevention of muscle damage of claim 1, wherein the ginseng extract is a concentrate concentrated under a reduced pressure comprising a non-saponin-based compound,
the ginseng extract is separated into water layer by concentrating the ethanol extract of the ginseng under a reduced pressure, and removing the ethanol,
butanol is added to the separated water layer and mixed, and then the mixture is separated into an upper layer and a lower layer,
each of the upper layer and the lower layer is separated into a form concentrated under the reduced pressure, and
the lower layer separated and concentrated under the reduced pressure is a water layer comprising a non-saponin-based compound.

4. The composition for stimulation of myogenesis and prevention of muscle damage of claim 1, wherein the muscle damage is selected from the group consisting of muscle strain, muscle rupture, muscle tearing, contusion, distortion, rotator cuff syndrome, and myositis.

5. A food composition comprising the composition for stimulating of myogenesis and prevention of muscle damage of claim 1.

6. A pharmaceutical composition comprising the composition for stimulation of myogenesis and prevention of muscle damage of claim 1.
